(19) United States
(12) Patent Application Publication
(10) Pub. No.: US 2005/0277847 A1
(43) Pub. Date: Dec. 15, 2005

(54) Sampling device

(75) Inventor: Jerome L. Belinson, Cleveland Heights, OH (US)

Correspondence Address: TAROLLI, SUNDHEIM, COVELL & TUMMINO L.L.P., 526 SUPERIOR AVENUE, SUITE 1111, CLEVEVLAND, OH, 44114, US

(73) Assignee: The Cleveland Clinic Foundation

(21) Appl. No.: 11/148,072

(22) Filed: Jun. 8, 2005

Related U.S. Application Data
(60) Provisional application No. 60/578,329, filed on Jun. 9, 2004.

(51) Int. Cl. A61B 10/00

(52) U.S. Cl. 600/569

(57) ABSTRACT

An apparatus for use in obtaining a woman's reproductive system includes a sheath and a sampler which is telescopically received in the sheath. A proximal end portion of the sampler includes a handle having a bearing surface which tapers in a direction away from the handle. A distal end portion of the sampler includes a brush having bristles with end portions arranged in a sphere. When the sampler is an extended position, the brush is spaced from a distal end portion of the sheath and the bearing surface on the handle is disposed in engagement with a tapered bearing surface on the sheath.

### RELATED APPLICATION

The benefit of the earlier filing date U.S. Provisional Patent Application Ser. No. 60/578,329 filed Jun. 9, 2004 by Jerome L. Belinson and entitled Sampling Device is hereby claimed. The disclosure in the aforementioned provisional Patent Application Ser. No. 60/578,329 filed Jun. 9, 2004 is hereby incorporated herein in its entirety by this reference thereto.

### BACKGROUND OF THE INVENTION

The present invention relates to an apparatus for use in obtaining a sample from a woman's reproductive system.

Sampling of cervical material is essential for the early detection and successful treatment of cervical cancer. Typically, a physician obtains the sample from a patient in what can be an embarrassing and inconvenient procedure. Many women avoid physician-conducted sampling, despite the recognized benefits of early detection. For many women, especially in the developing world, economic, geographic, and cultural barriers stand in the way of physician-conducted sampling.

Patient self-sampling may be a way to overcome these barriers, especially in light of recently identified viral and molecular markers. Sampling devices for self-sampling can be widely distributed by physicians and/or health workers, who may also train women in how to self-sample. Women can then collect their own samples privately. The women can then return, at their convenience, the sample to a physician, lab, or some central collection site. To date, no device for self-sampling has been shown to be as accurate as sampling by a health care provider.

A number of sampling devices have been developed to facilitate self-sampling and improve the sensitivity and acceptability of the technique. The main objective of these devices is to create a sampling device that is not intimidating, is easy to use, and which effectively collects the cervical material needed for screening.

A known sampling device includes a cylindrical sheath, which encloses a sample collector. The sheath can be straight or curved and it can be made from a variety of materials including cardboard, paper and plastics. Further, the sheath may be configured to approximate the geometry of a vagina. A slight curve in the sheath optimally matches this geometry but may not be necessary. Some known sampling devices are disclosed in U.S. Pat. Nos. 4,227,537; 5,445,164; 5,456,265; and 6,352,513.

The configuration and composition of a sample collector is important. A sample collector must be made of a material abrasive enough to collect target cervical material, while at the same time not being so abrasive that discomfort discourages use. A sponge and/or a brush may be used.

### SUMMARY OF THE INVENTION

The present invention relates to a new and improved apparatus for use in obtaining a sample from a woman's reproductive system. The apparatus includes a sheath having distal and proximal end portions. A sampler having distal and proximal end portions is utilized in association with the sheath. The proximal end portion of the sampler may include a handle having a bearing surface which tapers in a direction away from the handle.

The distal end portion of the sampler may include a brush. The brush may have bristles with end portions which are arranged in a configuration corresponding to the configuration of at least a portion of a sphere. The brush is connected with the handle by a shaft having a length which is the greater than the length of the sheath. When the apparatus is in a retracted condition prior to insertion into a woman's reproductive system, a portion of the shaft extends outward from the proximal end portion of the sheath so that the handle is spaced from the proximal end portion of the sheath and the brush is at least partially enclosed by the sheath. After the sheath has been inserted into the woman's reproductive system, the sampler is axially movable relative to the sheath from a retracted position to an extended position to move the brush out of the sheath. When the sampler is in the extended position, a tapered bearing surface on the handle may engage the tapered bearing surface on the sheath to facilitate rotation of the sampler relative to the sheath.

### BRIEF DESCRIPTION OF THE DRAWINGS

The foregoing and other features of the invention will become more apparent upon a consideration of the following description taken in connection with the accompanying drawings wherein:

FIG. 1 is a schematic pictorial illustration of an apparatus constructed in accordance with the present invention, the apparatus is illustrated in an extended condition in which a brush extends from a sheath and is rotatable relative to the sheath by manually rotating a handle;

FIG. 2 is a schematic sectional view further illustrating the construction of the apparatus of FIG. 1;

FIG. 3 is an enlarged illustration of a brush used in the apparatus of FIGS. 1 and 2;

FIG. 4 is an enlarged view of a brush shaft and handle used in the apparatus of FIG. 1;

FIG. 5 is a plan view of a sheath used in the apparatus of FIG. 1;

FIG. 6 is a schematic illustration, generally similar to FIG. 1, illustrating the manner in which the brush is enclosed by the sheath when the apparatus is in a retracted condition;

FIG. 7 is a schematic pictorial illustration of a second embodiment of the apparatus for use in obtaining a sample from a woman's reproductive system, the apparatus being illustrated in a retracted condition;

FIG. 8 is a schematic illustration, generally similar to FIG. 7, of the apparatus in an extended condition; and

FIG. 9 is an enlarged fragmentary sectional view illustrating a manner in which a tapered bearing surface on a handle of the apparatus of FIGS. 7 and 8 engages a tapered bearing surface on the sheath.

### DESCRIPTION ONE SPECIFIC PREFERRED EMBODIMENTS OF THE INVENTION

General Description

An apparatus 20 for use in obtaining a sample from a woman's reproductive system is illustrated in FIG. 1. The apparatus 20 is a self-sampler for use by a woman to obtain a cervical cell sample. However, it should be understood that the apparatus 20 may be used by medical personnel to obtain a cervical cell sample.

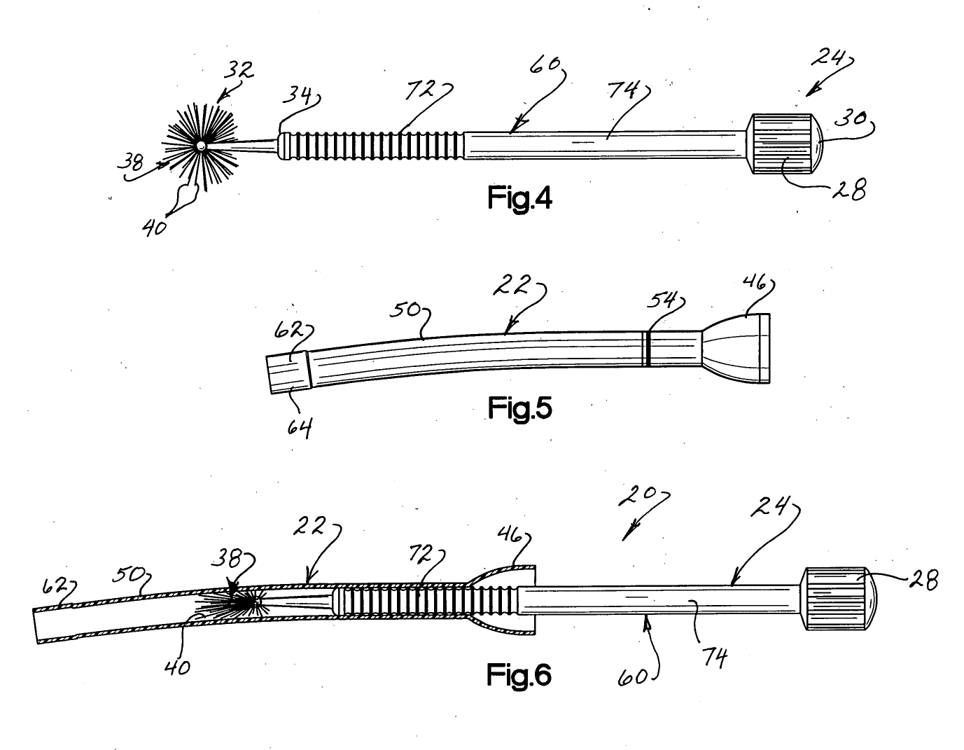

The apparatus 20 includes a tubular sheath 22 (FIGS. 1, 2, 5, and 6) which partially encloses a sampler 24 (FIG. 4). The sampler 24 includes a handle 28 which is disposed at a proximal end portion 30 of the sampler 24 (FIG. 4). A sample collector 32 (FIG. 3) is disposed at a distal end portion 34 (FIGS. 3 and 4) of the sampler 24.

It is contemplated that the sample collector 32 may have many different constructions. However, the illustrated sample collector 32 is a brush 38 having soft bristles 40 arranged in a spherical configuration. The spherical configuration of the brush 38 facilitates engagement of the brush with a lower end portion of the cervix at the outlet from the cervical canal of a woman's reproductive system. Although the illustrated sample collector 32 is a brush having a spherical configuration, it should be understood that the sample collector 32 may have a different construction if desired. For example, the brush 38 may have a non-spherical configuration. Alternatively, the sample collector 32 may be formed of an open or closed cell foam. If desired, the sample collector 32 may be coated with a material which enhances the collection of cells from the cervical area of a woman's reproductive system.

When the apparatus 20 is to be utilized to obtain a sample from a woman's reproductive system, the apparatus 20 is operated to the retracted condition of FIG. 6. When the apparatus 20 is the retracted condition, the brush 38 is enclosed by the sheath 22. The handle 28 is spaced from a proximal end portion 46 of the sheath 22.

While the apparatus 20 is in the retracted condition, the sheath 22 is gently inserted into the vagina of a woman's reproductive system. The sheath 22 has a generally cylindrical configuration and can be flexed to conform to the configuration of the woman's vaginal tract as the sheath 22 is inserted. Tubular sheath 22 has a smooth, generally cylindrical, outer side surface 50 which is formed of a material having a low coefficient of friction to minimize irritation of delicate tissues as the sheath is inserted into the vaginal tract. A circular band or marker 54 (FIG. 5) is provided on the sheath 22 to indicate the desired depth of insertion of the apparatus 20. When the apparatus 20 has been inserted into a woman's vaginal tract to a depth corresponding to the indicia 54, insertion of the sheath is interrupted.

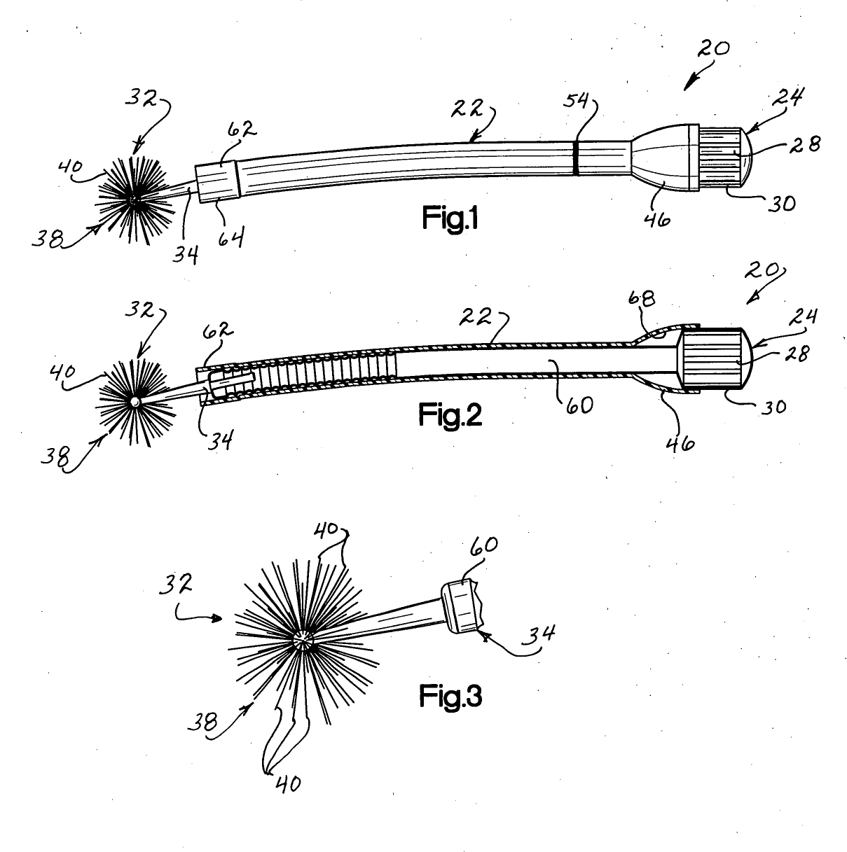

The sampler 28 is then telescopically moved from the retracted position of FIG. 6 to the extended position of FIGS. 1 and 2. To accomplish this, a woman in whose vaginal tract the sheath 22 is disposed, grips the proximal end portion 46 of the sheath with one hand and gently applies force against the handle 48 with the other hand. The force applied against the handle 28 is transmitted through a shaft 60 to the brush 38. As the handle 28 is moved toward the proximal end portion 46 of the sheath 22, the brush 38 moves from the retracted position shown in FIG. 6 toward a distal end portion 62 of the tubular sheath 22.

Continued movement of the handle 28 toward the proximal end portion 46 of the sheath causes the brush 38 to emerge from the distal end portion 62 of the sheath and move to the extended position shown in FIGS. 1 and 2. As this occurs, the brush 38 expands from the contracted condition of FIG. 6 to the expanded condition of FIG. 3. As the bristles 40 of the brush move to the expanded condition of FIG. 3, the flexible bristles form a spherical array at the distal end portion 34 of the sampler 24.

At this time, the soft bristles 40 of the brush 38 engage the woman's reproductive system at a location adjacent to the lower end of the uterus. One hand of the woman then rotates the handle 28 while the other hand holds the proximal end portion 46 of the sheath 22. This results in the soft bristles 40 being wiped across the lower end of the cervix at the location where the cervical canal opens into the vagina. The bristles 38 are soft and flexible and do not irritate the delicate tissues of the woman's reproductive system as the bristles are wiped across the tissue.

As the handle 28 is rotated and the bristles 40 of the brush 38 wipe across tissues of the woman's reproductive system, cervical cell samples adhere to the bristles 40 of the brush 38. Once the brush 38 has been rotated to collect a cervical sample, the brush is moved from the extended position of FIGS. 1 and 2 back to the retracted position of FIG. 6. To accomplish this, the woman grips the proximal end portion 46 of the sheath 22 with one hand and pulls the handle 28 away from the proximal end portion 46 of the sheath. As this occurs, the bristles 40 on the brush 38 move into the sheath 22.

Continued movement of the handle 28 away from the proximal end portion of the sheath 46 results in the sampler 24 moving to the retracted position of FIG. 6. At this time, the brush 38 is enclosed by the sheath 22 and the cervical cells which have adhered to the bristles 40 of the brush 38 are protected against being dislodged from the bristles. The woman then gently pulls on the proximal end portion 46 of the sheath to move the sheath out of her vagina. As this occurs, there is no relative movement between the sheath 22 and sampler 24 so that the brush 38 remains in the position shown in FIG. 6 relative to the sheath.

Once the apparatus 20 has been removed from the woman's reproductive system, the sampler 24 is again moved from the retracted position at FIG. 6 back to the extended position of FIGS. 1 and 2. The brush 38 is then immersed into a preservative solution, such as a Digene™ preservative solution, and agitated. This results in the dislodgement of the cervical cells from the bristles 40 of the brush 38 into the preservative solution. The preservative solution is then sent to a suitable laboratory for analysis.

Sheath

The sheath 22 has a tubular construction and is formed of a polymeric material. The sheath 22 has a permanent bend which, to some extent at least, corresponds to the non-linear configuration of the central axis of a woman's vaginal tract. The sheath 22 is flexible so it can be readily deflected to conform to the curvature of a vaginal tract into which it is inserted. The sheath 22 is formed of a polymeric material.

The sheath may be composed of linear low density polyethylene if desired. However, the sheath may be formed of other materials, for example, cardboard or paper. If the sheath 22 is made with an absorbent material, the sheath may be coated with a non-absorbent slick material to increase comfort and to protect against the sheath absorbing material and a sample adhering to the brush 48. If desired, a sleeve 64 may be provided on the distal end portion 62 of the sheath 22.

The proximal end portion 46 of the sheath 22 is enlarged and receives a portion of the handle 28 when the sampler 24 is in the extended position illustrated in FIGS. 1 and 2. The enlarged proximal end portion 46 of the sheath 22 has a tapered inner side surface 68 which functions as a bearing surface for the handle 28. The inner side surface 68 (FIG. 2) of the proximal end portion 46 of the sheath 22 maintains the handle 28 in alignment with a longitudinal central axis of the sheath 22 during rotation of the handle.

The sheath 22 has an overall length of about 9 to 20 cm. At locations between the proximal and distal end portions 46 and 62 of the sheath 22, the sheath is a tubular cylinder having an outside diameter of about 0.75 to 2.5 cm. The inner diameter of this portion of the sheath 22 is about 0.25 to 1.5 cm. The marker 54 may be located at approximately 9 to 13 cm from the distal end portions 62. Of course, the specific location marker 54 will depend upon the overall extent of the sheath 22. It is believed that it may be desired to have the sheath relatively short so that the apparatus 20 is not intimidating to a woman who is to use the apparatus.

Sampler

The sampler 24 is telescopically received in the sheath 22. By pushing on the handle 28, the sampler 24 can be moved from the retracted position of FIG. 6 to the extended position of FIGS. 1 and 2. Similarly, by pulling on the handle 28, the sampler 24 can be moved from the extended positions of FIGS. 1 and 2 to the retracted position of FIG. 6.

The cylindrical shaft 60 connects the handle 28 to the brush 38. The shaft 60 is a tubular construction and includes a flexible distal end portion 72 (FIG. 4) and a proximal end portion 74. The proximal end portion 74 of the shaft 60 has a straight tubular configuration. The proximal end portion 74 of the shaft 60 can be deflected sideways from the illustrated linear configuration by the application of a sideward force against the proximal end portion of the shaft 60.

The distal end portion 72 of the shaft 60 is formed by a flexible cylindrical tubular bellows. This allows the distal end portion 72 of the shaft 60 to readily flex upon the application of a relatively small force to the brush 38. The distal end portion 72 and proximal end portions are both hollow and are disposed in a coaxial relationship with each other and with the handle 28 and brush 38.

The bristles 40 of the brush 38 are soft and flexible so as not to irritate delicate body tissues. The bristles may be made of a suitable polymeric material, for example, white nylon fill. The bristles may have any desired length. In one specific instance, the bristles had a length of 1.5 to 3.0 cm and did not taper. In this specific instance, each bristle had a diameter of approximately 0.01 to 0.02 cm. When the sampler 24 is in the retracted position of FIG. 6, the bristles collapse to have an overall diameter of 0.5 to 2.0 cm.

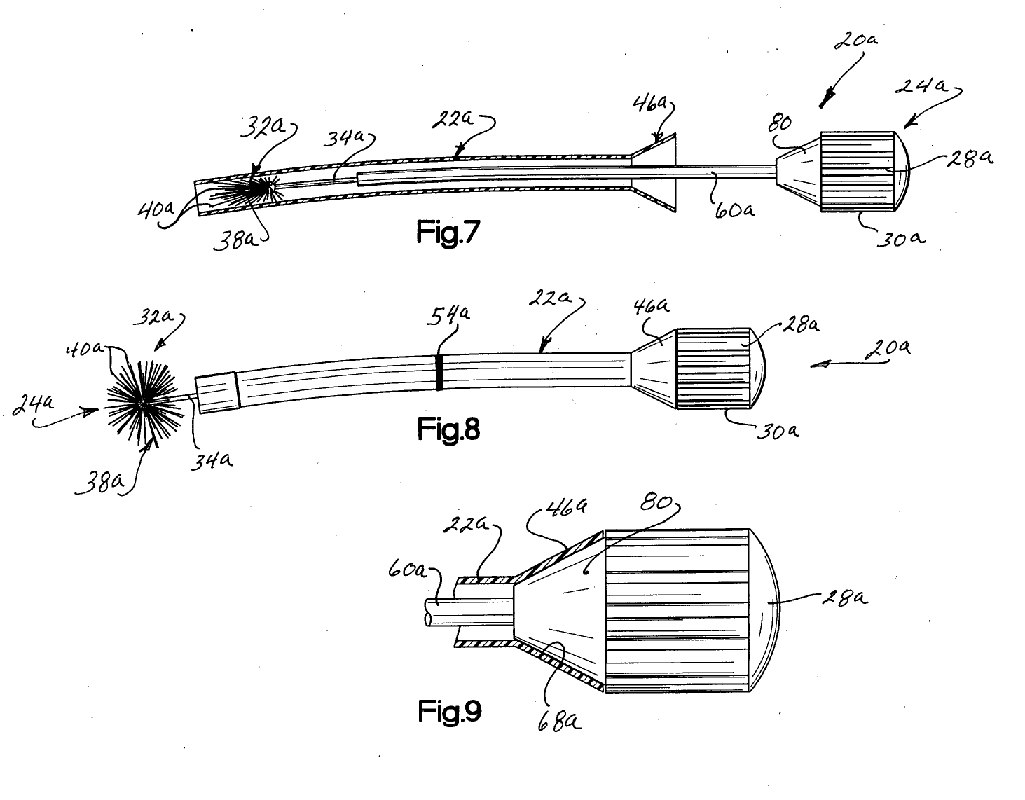

Embodiment of FIGS. 7-9

The embodiment of the invention illustrated in FIGS. 7-9 is generally similar to the embodiment of the invention illustrated in FIG. 1-6. Therefore, similar numerals will be utilized to designate similar components, the suffix letter “a” being added to the numerals of FIGS. 7-9 to avoid confusion.

An apparatus 20a includes a sheath 22a in which a sampler 24a is telescopically received. The sampler 24a is movable relative to the sheath between the retracted position of FIG. 7 and the extended position of FIG. 8. The apparatus 20a is used as a self-sampler by a woman to collect a cervical cell sample in the same manner as previously described in conjunction with the embodiment of the invention illustrated in FIGS. 1-6. Although it is believed that the apparatus 20a will be used by a woman as a self-sampler, the apparatus may be used by a person other than the woman to obtain a cervical cell sample from the woman.

The sampler 24a includes a handle 28a at a proximal end portion 30a of the sampler. In addition, the sampler 24a includes a sample collector 32a at a distal end portion 34a of the sampler. In the illustrated embodiment of the invention, the sample collector 32a is a brush 38a having soft bristles 40a.

The apparatus 20a is inserted into a woman's vaginal tract with the apparatus in the retracted condition of FIG. 7. When the sheath 22a has been inserted to a distance corresponding to a marker 54a, movement of the sheath 22 into the vaginal tract is interrupted. The apparatus 20a is then operated from the retracted condition of FIG. 7 to the extended condition of FIG. 8. As this is done, the woman grips a proximal end portion 46a of the sheath and pushes the handle 28a gently toward the proximal end portion 46a of the sheath. As this occurs, the brush 38a emerges from the sheath at a location adjacent to a lower end portion of the cervical canal.

The woman then rotates the handle 28a relative to the sheath 22a. This causes the soft bristles 40a of the brush 38a to wipe across the lower end of the cervix and to collect cells from the cervical area.

Once the brush 38a has been rotated to collect cervical cells on the bristles 40a, the apparatus 20 is operated back to the retracted condition of FIG. 7. This is accomplished by pulling the handle 28a away from the proximal end portion 46a of the sheath 22a. Thereafter and while holding the handle 28a against movement relative to the sheath 22a, the sheath is pulled from the woman's vaginal tract. The apparatus 20 is then operated back to the extended position of FIG. 8 and the brush 38a is immersed into a preservative solution, such as a Digene (trademark) solution. The handle 28a is then rotated and the brush moved in the solution to wash cervical cells from the brush. The preservative solution, with the cervical cells therein, is then sent to a laboratory for testing.

The handle 28a has a bearing surface 80 which tapers toward the distal end portion 34a of the sampler 24a. The bearing surface 80 is disposed in a coaxial relationship with the shaft 60a. The bearing surface 80 has a configuration corresponding to the configuration of the frustrum of a right circular cone.

When the apparatus 20a is in the extended condition of FIG. 8, the bearing surface 80 on the handle 28a is disposed in engagement with the inner side or bearing surface 68a on the proximal end portion 46a of the sheath 22a. Engagement of the two bearing surfaces 68a and 80 positions the brush 38a relative to the sheath 22a when the apparatus 20a is in the extended condition in FIG. 8. In addition, engagement of the bearing surfaces 68a and 80 results in the handle 28a being positioned relative to the sheath 22a. The bearing surfaces 68a and 80 cooperate to align the handle 28a and shaft 60a with the sheath 22a during rotation of the handle.

### CONCLUSION

In view of the foregoing description, it is apparent that the present invention provides a new and improved apparatus 20 for use in obtaining a sample from a woman's reproductive system. The apparatus 20 includes a sheath 22 having distal and proximal end portions 62 and 46. A sampler 24 having distal and proximal end portions 34 and 30 is utilized in association with the sheath 22. The proximal end portion 30 of the sampler 24 may include a handle 28 having a bearing surface 80 which tapers in a direction away from the handle.

The distal end portion 34 of the sampler 24 may include a brush 38. The brush 38 may have bristles 40 with end portions which are arranged in a configuration corresponding to the configuration of at least a portion of a sphere. The brush 38 is connected with the handle 28 by a shaft 60 having a length which is the greater than the length of the sheath 22. When the apparatus 20 is in a retracted condition prior to insertion into a woman's reproductive system, a portion of the shaft 60 extends outward from the proximal end portion 46 of the sheath 22 so that the handle is spaced from the proximal end portion of the sheath and the brush is at least partially enclosed by the sheath. After the sheath 22 has been inserted into the woman's reproductive system, the sampler 24 is axially movable relative to the sheath from a retracted position to an extended position to move the brush 38 out of the sheath 22. When the sampler 24 is in the extended position, the tapered bearing surface 80 on the handle may engage a tapered bearing surface 68 on the sheath 22 to facilitate rotation of the sampler 24 relative to the sheath 22.

1. An apparatus for use in obtaining a sample from a woman's reproductive system, said apparatus comprising:
a sheath having distal and proximal end portions, at least a portion of said proximal end portion of said sheath having a radially outwardly flaring configuration, said sheath having a tubular construction with a circular cross section between said proximal and distal end portions, said distal end portion of said sheath having a circular cross sectional configuration; and
a sampler having distal and proximal end portions, said proximal end portion of said sampler including a handle having a bearing surface which tapers in a direction away from said handle, said distal end portion of said sampler including a brush connected with said handle by a shaft having a length which is grater than a length of said sheath, a portion of said shaft extends outward from the proximal end portion of said sheath so that said handle is paced from said proximal end portion of said sheath when said sampler is in a retracted position with said brush at least partially enclosed by said sheath, said sampler being axially movable relative to said sheath from said retracted position to an extended position under the influence of force manually applied to said handle, a portion of said shaft extends outward from the distal end portion of said sheath so that said brush is spaced from said distal end portion of said sheath when said sampler is in an extended position with said tapered bearing surface on said handle disposed in engagement with a bearing surface which is disposed on said proximal end portion of said sheath and which tapers in a direction toward said distal end portion of said sheath.

2. An apparatus as set forth in claim 1 further including a mark disposed on said sheath to indicate a depth to which said sheath is to be inserted into an opening in the woman's body with the sampler in the retracted position.

3. An apparatus as set forth in claim 1 wherein said bristles of said brush are arranged in a spherical configuration.
